(19) United States
(12) Patent Application Publication
(10) Pub. No.: US 2007/0100290 A1
(43) Pub. Date: May 3, 2007

(54) Positioning Aid For Fitting A Needle On An Injection Device

(76) Inventors: Frank Schiffmann, Burgdorf (CH); Reto Michel, Derendingen (CH); Kurt Weber, Bolken (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/531,639

(22) Filed: Sep. 13, 2006

(30) Foreign Application Priority Data
Sep. 14, 2005 (DE) 10 2005 043 805.9

(51) Int. Cl. A61M 5/32; A61M 5/00

(52) U.S. Cl. 604/198; 604/110; 604/192; 604/263

(57) ABSTRACT

A positioning mechanism and method for fitting a needle on an injection device with an associated needle cover wherein the needle cover has at least one positioning element for guiding or positioning a needle to a pre-defined position relative to the injection device.

### CROSS-REFERENCE TO RELATED APPLICATION

This application claims priority to German Application No. 10 2005 043 805.9, filed on Sep. 14, 2005, the entire content of which is incorporated herein by reference.

### BACKGROUND

The present invention relates to devices for administering, delivering, injecting or dispensing substances, and methods of making and using such devices. More particularly, it relates to a positioning and/or guiding mechanism and, more particularly, a centering aid for fitting a needle or cannula on an injection device or on an ampoule, as well as to an injection device with a needle guard for an injection needle, which can be or is already fitted on a front, distal end of the injection device, and has a centering aid. The injection device may be used for self-administering an injectable product, for example insulin, a growth hormone or an osteoporosis medicament. In particular, the injection device may be of the type of device commonly known as an injection pen.

In the case of patients with fine motor-related limitations, such as patients suffering for osteoporosis for example, it can be difficult to fit an injection needle retained in a needle holder precisely axially on an injection device and screw it on, if necessary. A self-centering ring of a needle thread of a needle holder on support does not help because the needle projecting out of the needle holder or support in the proximal direction as the injection device is being assembled pierces the septum of an ampoule before the needle thread moves into engagement, which means that if an injection needle cannula is not correctly fitted, the rear part of the cannula projecting out from the needle holder may become bent or the ampoule stopper may be pierced at an angle. Consequently, if a needle is incorrectly fitted, there may be no passage through the cannula to the medicament chamber, rendering dispensing impossible. In such a situation, excessive pressure may also occur in the ampoule when attempting to administer an injection.

The injection needle of an injection device poses a risk of injury, for example when changing the needle or during handling if fitting in the correct position takes longer, and in many patients who administer the product by injection themselves instils fear (needle phobia). These problems can lead to incorrect injection or at least a sub-optimal injection. To reduce fear of the injection needle, injection devices are often provided with a needle screening cover which, in a protective position, surrounds the needle so that the user of the injection device can not see the needle. Such needle screening covers may be held in the protective position by a mechanical spring. To administer an injection, the user places the injection device on the injection site on the skin and presses it against the skin so that the needle screening cover moves against the force of the spring out of the protective position into an injection position, and as this happens the injection needle is simultaneously introduced into the body tissue. As it is pulled out of the body tissue, the needle screening cover is automatically returned to the protective position due to the rebounding force of the spring.

In addition to needle screening covers, needle guards are also known for injection devices. Typically, these guards are not intended purely as a means of concealing the injection needle, at least not primarily, but are provided as a means of protecting the user against injuries due to the injection needle. Typically, the needle guard is locked in said protective position and can not yet be moved in the proximal direction solely by a pressing force acting in the proximal direction, but can only be released by breaking or by an additional hand grip by which a lock is actively applied in the protective position. In principle, such needle guards may afford a view of the needle, or alternatively, may also have the property of blocking sight of it in addition to fulfilling the function of access protection.

U.S. Pat. No. 6,033,387 discloses a needle guard for an injection device in the form of a simple syringe. The needle guard completely surrounds the syringe and forms an outer housing for the entire syringe. When the needle guard is moved by hand into its protective position after an injection, it can not be moved back out of the protective position, at least not until the syringe has been moved back into a fitting position. In particular, there is no injection position for the needle guard from which it automatically moves in the distal or forward direction into the protective position when pressure is released.

Injection devices of the type to which the present invention relates typically have a container holder, in which a container filled with the product or substance to be injected is inserted, and this is replaced with a new container after use. In many cases, the container is an ampoule filled by the product manufacturer, which is closed by an axially displaceable plunger at one end and by a seal element at the other end. Since such injection devices are designed for a longer service life, using several product containers one after the other, and the injection needle may also be replaced from time to time, for example together with the container. To this end, the injection needle is attached to a needle holder, which is connected to the injection device. When the needle holder is connected to the injection device, the injection needle pierces the seal element of the container by means of a proximal or rearward facing needle tip, establishing the flow connection between the interior of the container and the injection needle.

The disadvantage of needle covers for devices of this type, such as disclosed in patent specifications DE 197 55 125 A1 and DE 100 09 816 A1 for example, is that they can not be connected to the injection device until the needle holder has been connected to the injection device. This means that the user of the injection device has to attach the needle cover with the injection needle exposed and, therefore, has to work with the injection needle exposed when fitting the needle cover.

Patent specification DE 103 48 185 A1, owned by the owner of the present application, discloses an injection device with a needle cover, which is provided on a distal or forward end of an injection device and can be pushed into the injection device.

As used herein, the term “needle cover” is intended to encompass the terms needle guard, needle screen, needle screening cover, needle guide and the like and, if no comment is made in respect of protective or visual screening functions, it should be interpreted as meaning that it may include a needle guide or needle positioning element and optionally also a needle screening cover or needle access guard. Combinations of needle access guards and needle screening covers and the like are intended to be emcompassed as well.

### SUMMARY

An object of the present invention is to provide a positioning mechanism and a method for correctly mounting or fitting an injection needle relative to an injection device or a container such as an ampoule, including mounting or fitting the needle so that it is selectively positioned, piercingly positioned or centered.

In one embodiment, the present invention comprises a positioning mechanism for fitting a needle on an injection device with an associated needle cover wherein the needle cover has at least one positioning element for guiding or positioning a needle to a pre-defined position relative to the injection device.

In one embodiment, the present invention comprises a method for fitting a needle on an injection device with an associated needle cover wherein the needle cover has at least one positioning element for guiding or positioning a needle to a pre-defined position relative to the injection device.

In one embodiment, the present invention comprises a positioning mechanism for placing a needle in a piercing position on an injection device, the positioning mechanism comprising one of a needle guide, a needle cover, a needle holder or a protective cap for the needle which is or can be detachably mounted on the injection device, and at least one positioning element associated with the one of a needle guide, needle cover, needle holder or protective cap adjacent to an end thereof, whereby the needle is guided and/or placed in the piercing position by the positioning element.

In one embodiment, the present invention comprises a method for fitting a needle on an injection device or on an ampoule in a positioned manner, the method comprising the steps of providing a needle holder carrying a needle or a needle, providing a positioning element associated with a needle cover connectable to the injection device or to the ampoule, and positioning the needle relative to the needle cover and, thus, relative to the injection device or the ampoule. The method further comprises moving the needle cover toward the injection device or the ampoule, the needle holder or the needle thus guided onto the injection device or the ampoule in a positioned manner.

In one embodiment, the present invention comprises an injection device comprising a housing for receiving an injectable product or a container containing an injectable product, a needle holder comprising an injection needle connectable to the injection device or container by the needle holder, and a needle cover carried by the housing such that it can be moved against a restoring force from a protective position, beyond an injection position into a fitting position, wherein the needle cover extends beyond a needle tip of the injection needle in the protective position and is short of the needle tip in the injection position and affords free access to the needle holder in the fitting position, such that the needle holder can be held by a protective sleeve and can be connected to or released from the housing, the needle cover comprising a positioning aid at one end onto which an outer protective cap surrounding the injection needle can be fitted and oriented relative to the injection device or container.

In one embodiment, a positioning mechanism for producing a centered or positioned attachment of a needle or cannula on an injection device or a container, such as an ampoule which is also inserted in the injection device or is formed by the latter, has a needle cover which is disposed by preference on the distal or forward end of the injection device so that it can be displaced in the axial direction of the injection device. For the purpose of the present invention, the needle cover has a positioning element on a needle or a fitting end for a needle holder, in other words at the distal or forward end, by which a needle or a needle holder in which the needle is retained, being welded, cast or glued in it for example, can be positioned before or as the needle holder or needle fitted on the needle cover is displaced relative to the injection device. Consequently, a needle or a needle holder already provided with a needle guard by the manufacturer in the form of one or more protective caps for example, can be correctly positioned or centered by the needle cover permanently connected to the injection device as it is pushed in the proximal or rearward direction of the injection device to pierce the center of a septum of an ampoule by the needle or cannula. For example, in some embodiments, the needle cover of the injection device may be designed so that the edge of an outer protective cap, which is placed on a needle holder above an inner protective cap, is centered or positioned relative to the injection device by the positioning element of the needle cover, which may be funnel or generally cone-shaped. Accordingly, as it is being fitted and prior to piercing the septum of a stopper closing a container of a substance to be administered, the needle can be oriented to a piercing position. As the needle holder is fitted on the injection device, the needle cover moves with it so that the needle is continuously guided and centered by the needle cover.

In some embodiments, an injection device in accordance with the present invention comprises a housing, which constitutes a mounting for a container containing an injectable product or itself directly forms such a container. The injection device has a conveying mechanism for the product, a needle holder with an injection needle and a needle cover, on or by means of which a positioning element is formed. In some preferred embodiments, the needle holder may be designed so that it can be or is connected to the housing, irrespective of whether the housing itself forms the product container or the product container is a separate, replaceable container which the housing accommodates. In principle, in the case of a product container accommodated by the housing, it would also be conceivable for the needle holder to be connected or connectable to such a container, in which case the needle itself can then be fitted and positioned by the needle cover in accordance with the present invention. The injection needle is held by the needle holder so that is connected to the interior of the container establishing a flow communication when the needle holder is connected to the container or to the housing. When the needle holder is in the connected state, the product is dispensed from the container through the injection needle by the conveying action of the conveying mechanism.

In some preferred embodiments, the positioning element of the needle cover comprises at least one surface element, which is designed so that a plane extending through the needle guide axis, such as a longitudinal axis of the injection device or ampoule or through the needle piercing line for example, intersects the surface element so that the straight intersection line or intersection curve extends towards the needle piercing axis, from the distal or forward end to the proximal or rear of the injection device. In some preferred embodiments, the surface element provided on the needle guide or needle cover may be designed so that it extends around a part or the entire edge of the needle cover, which may be cylindrical, and is funnel-shaped or conically converging.

In some embodiments, the positioning element of the needle cover may project radially inward and/or radially outward from the needle guard or needle cover, thereby enabling a centering action or at least a guiding action, on a fitted needle or a fitted needle holder, into a desired position, suitable for the needle to pierce a septum.

In some preferred embodiments, an oblique surface provided on the needle cover as a positioning element has a gradient, as measured in section, of 30° to 60°, 40° to 50°, or 45°, with reference to an axial direction of the injection device.

In some embodiments, the needle holder may be of the type of known needle holders, where the injection needle is connected to the needle holder by a middle needle portion, with a distal or forward needle portion projecting beyond the needle holder and with a proximal or rear needle portion projecting from the proximal or rear end, i.e. projecting out from the end of the needle holder connected to or connecting the housing or the container.

In some embodiments, the needle guard or needle cover is mounted so that it can be moved backwards and forwards from the housing in the distal and proximal directions. The needle cover is biased in the distal direction by a rebound force. Under the effect of the rebound force, it assumes a protective position in which it projects in the distal or forward direction beyond the distal or forward tip of the injection needle when the needle holder is connected to the housing or the container. The needle cover can be moved in the proximal direction against the rebound force, out of the protective position as far as an injection position, and in the proximal direction beyond the injection position into a fitting position. In the injection position, the distal, forward or injection tip of the injection needle projects beyond the distal end of the needle cover. To move the needle cover in the proximal direction, a force has to be applied to it in the direction opposite the rebound force and the rebound force overcome. Typically, such a force acts when the injection needle is introduced into a body tissue, in other words, due to the resistance of the tissue surface against which the needle cover is pressed during the injection. Once the force acting in the proximal direction in the injection position is released, the needle cover automatically moves back in the distal direction under the effect of the rebound force. To this extent, only the equilibrium of the forces prevails in the injection position, namely the rebound force and the counteracting force. The injection position is therefore not fixedly defined beforehand, but depends on the penetration depth of the injection needle during the injection. However, it is defined to the extent that the injection needle is only able to penetrate the tissue by means of its distal needle portion projecting beyond the needle holder during the injection. During the injection, therefore, the distal end of the needle cover and the distal end of the needle holder are generally adjacent or in the same plane relative to the needle length.

In some embodiments, the needle cover is displaceable still further beyond the injection position against the rebound force as far as a fitting position. In the fitting position, it affords free access to the needle holder so that the needle holder can be gripped and connected to the housing and/or released from the housing by hand. Accordingly, the needle cover can be connected to the housing before fitting the needle holder, in some instances when the injection device is assembled at the manufacturing stage. The needle cover does not have to be removed to replace the injection needle. In some preferred embodiments, it is an integral part of the injection device and can not be released from the injection device at all, i.e. from its housing, by the user, generally the patient who is administering the product himself, thereby reducing the risk of incorrect fitting. In particular, however, the risk of injury by the injection needle is reduced during fitting and dismantling the needle holder, for example when replacing the injection needle. In effect, the user does not have to fit a needle cover with the injection needle exposed.

In some embodiments, in the fitting position, the needle cover is so far behind the distal or front end of the needle holder in the proximal or rear direction that the user can take hold of the needle holder with the fingers comfortably from the side, i.e. transversely to the needle length, and carry out the necessary manipulations to fit and/or dismantle the needle holder.

As mentioned above, in one preferred embodiment, the housing serves as a mount for a product container which, in another preferred embodiment, can be replaced. To this end, the housing has a container holder, forming a housing chamber for the container. A stop for the container is provided in the housing chamber, against which the container is in contact in the distal direction. The container holder secures the container in the housing chamber to prevent movements transversely to the distal and proximal direction. The housing chamber is open at a proximal chamber end so that the container can be introduced into the housing chamber in the distal direction until it makes contact with the stop. However, it would also be possible for the housing chamber to be provided with the opening for the container at its distal end or, in principal, also at the side. The container holder is intrinsically dimensionally stable, i.e. not flexible, but is rigid.

In some embodiments the needle cover is supported against such a container holder in the proximal direction so that the rebound force acts between the needle cover and the container holder as an elastic force. In principle, instead of being supported directly on the container holder, the needle cover could also be supported on another housing part of the injection device which is connected to the container holder. The needle cover is guided in a sliding action on the container holder, i.e. the container holder forms a slide guide for the needle cover.

In some embodiments, a return mechanism generating the rebound force generates the rebound force in the form of an elastic force. The elastic force may be generated by a mechanical spring, e.g. a helical spring, which is biased forward at least in the fitting position and the injection position and, in some embodiments, also in the protective position, preferably based on pressure. In principle, it would also be possible for the elastic force to be generated by compressing a gas, e.g., air.

To make it easier to fit and/or dismantle the needle holder, in some embodiments, the needle cover is prevented from moving in the distal or forward direction in its fitting position by a locking mechanism acting between the needle cover and the housing, so that the user does not have to hold the needle cover against the rebound force. For locking purposes, the housing is provided with a first locking element and the needle cover is provided with a second locking element. When the needle cover is in the fitting position, the locking elements are locked in engagement, which holds the needle cover against the rebound force in the fitting position. Several first and several second locking elements, arranged respectively in pairs, may form a locking engagement. If several first and several second locking elements are provided, in some preferred embodiments, two first locking elements and two second locking elements are provided and a first one and a second one of the locking elements establish a locking engagement with one another respectively in pairs. Expediently, the two locking element pairs are disposed at diametrically opposite ends of the injection device by reference to an axial line. The locking engagement may be obtained purely on the basis of a frictional grip of the blocking elements, although in some preferred embodiments, the locking engagement is obtained on the basis of a positive connection, optionally a combination of frictional and positive connection.

In some embodiments, the injection device has an unlocking element which can be operated by the user, which releases the locking engagement when operated. The unlocking element may be provided directly in the form of a sleeve body, which may form the needle cover, so that the user can grip such a sleeve body in a sleeve portion directly in the fitting position, for example. The needle cover incorporates such a cover sleeve or a sleeve-shaped cover body, as well as an unlocking element which can be displaced relative to such a cover sleeve or such a sleeve-shaped cover body. If several locking element pairs are provided, such an unlocking element is provided for each pair. The unlocking element is provided in the form of a push button, which is depressed relative to the cover sleeve or the sleeve-shaped cover body to release the locking engagement.

In a preferred embodiment, at least one of the locking elements can be displaced out of the locking engagement against an elastic force and is automatically moved into the locking engagement due to the elastic force as the needle cover moves into the fitting position. The at least one locking element which can be displaced out of the locking engagement by means of the elastic force may itself be elastic. It is advantageously elastic in terms of its shape so that it simultaneously applies the elastic force to snap into the locking engagement, but at the same time has the stiffness needed to establish a positive connection. In some preferred embodiments, the elastic locking element is a flexible tongue with either a projecting cam or a recess or orifice, into which the other locking element projects in the locking engagement.

In some embodiments, the housing has a casing structure which surrounds at least an axial portion of the needle cover when the needle cover is in the fitting position. As it moves into the fitting position, the needle cover moves into the housing casing structure by means of at least said axial portion. The casing structure surrounds a distal portion of said container holder, leaving an annular chamber between the casing structure and the distal portion of the container holder, in which at least said axial portion of the needle cover is accommodated in its fitting position. The needle cover still projects into the housing casing structure and into the formed annular chamber by means of an axial portion in the protective position. In the fitting position, it is totally or almost totally accommodated in the annular chamber so that it terminates flush with the housing casing structure or so the distal end of the housing casing structure projects in the distal direction by only a short axial portion. The return mechanism may be accommodated in the annular chamber.

On the subject of the conveying mechanism, it should be pointed out that in a preferred embodiment, it comprises a plunger accommodated in the container, the stroke of which dispenses the product out of the container and through the injection needle. The plunger stroke is directed in one of the axial directions, e.g., in the distal direction.

In some embodiments, an injection device in accordance with the present invention is one which can be used more than once to administer a predefined product dose or several products doses which can be specifically selected. For this purpose, such an injection device has a dose metering mechanism, which engages with the conveying mechanism, e.g., in a mechanical engagement, which enables the product dose to be set by a dose metering movement of the dose metering mechanism, which can then be conveyed by means of the conveying mechanism when the conveying mechanism is operated. In a preferred embodiment of the conveying mechanism in the form of a reciprocating plunger-conveyor mechanism, this takes place on the basis of the length of the plunger stroke or, in the case of a plunger stroke which is always of identical length, the adjustment of a clearance distance between a rear face of the plunger and an end of a plunger rod axially facing the plunger rear face. During its conveying stroke, portions of the conveying mechanism initially travels the clearance distance as far as the plunger and then drives the plunger with it during the rest of the stroke.

In some embodiments, the present invention comprises a method of attaching a needle holder holding an injection needle to a distal or front end of an injection device as well as a method of releasing a needle holder holding an injection needle from the end of an injection device. The injection device is provided with a needle cover, which surrounds the injection needle to beyond a forward distal needle tip in a protective position when the needle holder is attached to the injection device, and which can be displaced out of the protective position against a rebounding elastic force in the direction towards the rear end of the injection device behind the front end of the needle holder into a fitting position.

When attaching the needle holder, the needle cover is moved out of the protective position into the fitting position in a first step, and a locking engagement is established by which the needle cover is retained in the fitting position against the elastic force. The locking engagement may be established automatically once the needle cover has reached the protective position. While the needle cover is in the locked protective position, the needle holder is attached to the end of the injection device. For example, it may be screwed onto the injection device. The needle holder is usually provided with a protective cap, which surrounds and protects the needle portion projecting beyond the needle holder and also protects the user from being pierced by the needle. When using such a needle holder, this protective cap is removed once the needle holder has been attached, so that the injection needle projects out uncovered. If another protective cap also surrounds the needle holder, this other protective cap is also removed, The locking engagement is then released so that the needle cover automatically moves back into the protective position under the action of the rebounding elastic force.

To release the needle holder, the needle cover is moved form the protective position into the fitting position in a first step and thus also into the locking engagement, as was the case with the fitting operation. With the needle cover in the fitting position, the needle holder may be released from the end of the injection device and disposed of. Before the releasing operation, when the needle cover is in the fitting position, a protective cap is attached to the needle holder, e.g., push or friction-fitted, to protect the user from being pierced by the needle.

By virtue of another aspect of the present invention, the invention also relates to a method of fitting a needle on an injection device or an ampoule so that it is centered otherwise selectively positioned, whereby a needle holder holding a needle or a needle directly is placed on a positioning element of a needle guide or needle cover and positioned by the positioning element relative to the needle guide or needle cover and hence relative to the injection device. The needle cover is pushed in the proximal direction of the injection device and the needle holder or needle thus positioned as it is guided onto the injection device or a container closure element of the ampoule.

In some embodiments, the needle or the needle holder is snap-fitted on the injection device, i.e. is latched onto the injection device by means of a catch element or a thread or is screwed onto it. An outer protective cap and optionally then an inner protective cap, if one is provided, can then be removed from the needle to expose the needle, whereupon the needle cover can be released so that, being a protective cover, it is pushed in front of the injection end of the needle, by the biasing force of a spring element for example, so that it can serve as a needle guard.

An injection can then be administered by means of the exposed needle inside the needle cover while simultaneously pushing back the needle cover, which is automatically pushed in front of the needle as the injection device is removed again and can also be latched in this position. A protective cap, such as the outer protective cap for example, can then be positioned as it is placed on the needle or the needle holder by the positioning element of the needle cover and screwed to the needle holder or connected by means of a catch connection to remove the needle holder from the injection device, by pulling it off or unscrewing it for example. As a result, the needle is not exposed during the described functional sequence other than for the injection itself, and is always surrounded by at least one protective cap both before and during removal of the needle from the injection device during the fitting operation, thereby reducing the risk of injury.

Although the present invention is described in the context of a needle cover, the needle cover incorporating at least one positioning element as proposed by the invention need not necessarily surround the needle completely. Instead, the needle cover of the present invention may also be purely a positioning mechanism for fitting the needle and/or for fitting a protective cap onto the needle in readiness for removing the needle from the injection device, in which case other elements may be provided to fulfil the function of a needle cover or a needle guard.

### BRIEF DESCRIPTION OF THE DRAWINGS

FIG. 1 is an exploded view of a distal (front) part of an injection device in accordance with the present invention with a first embodiment of a positioning or centering aid in accordance with the present invention,

FIG. 2 shows the injection device of FIG. 1 in the fitted state, including a fitted needle holder with a second embodiment of a centering aid, in which a needle cover of the injection device is in the fitting position,

FIG. 3 shows the distal part of the injection device of FIG. 1 with the needle cover in a protective position,

FIG. 4 shows the distal part of the injection device of FIG. 1 with the needle cover in an injection position;

FIG. 5A shows an embodiment of a positioning mechanism in accordance with the present invention in an initial position;

FIG. 5B shows the positioning mechanism illustrated in FIG. 5A as the cannula or needle effects a piercing action;

FIG. 5C shows the positioning mechanism illustrated in FIG. 5B after the needle has been fully fitted; and

FIG. 5D shows the needle fitted on an injection device after removing the outer and inner protective cap with the extracted needle cover.

### DETAILED DESCRIPTION

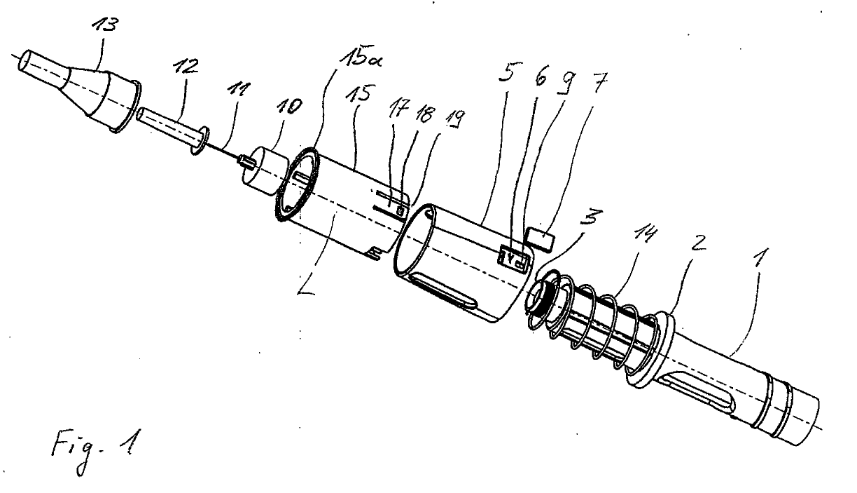

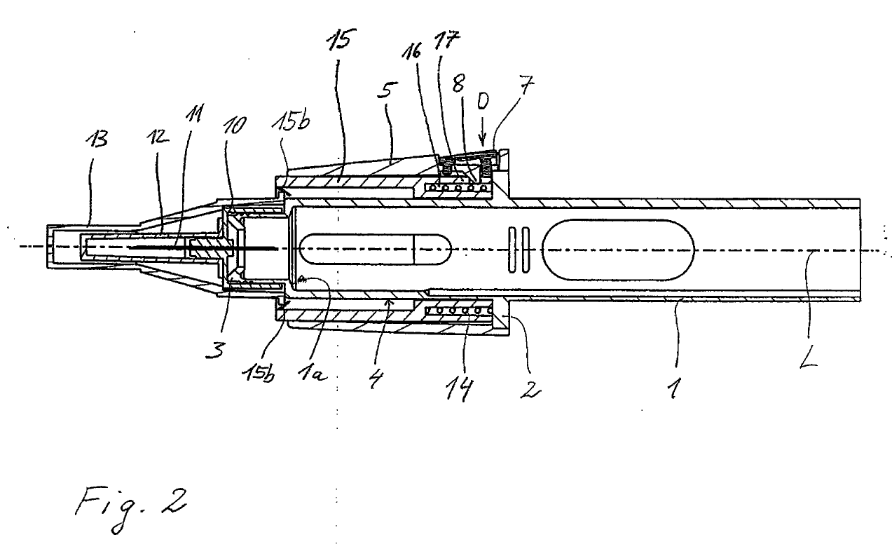

FIG. 1 is an exploded diagram illustrating a distal part of an injection device (the front, forward or needle-carrying end of the device) in accordance with one embodiment of the present invention, in which the separately made components of the injection device are aligned along a central longitudinal axis L of the injection device in a sequence suitable for assembly. FIG. 2 shows the same components in the assembled state, but with an alternative positioning element 15b to that illustrated in FIG. 1, projecting inwardly.

The distal part of the injection device comprises a container holder 1 and a casing structure 5, which form the distal part of a housing of the injection device in the assembled state, as may be seen from FIG. 2, for example. The distal or forward part of the injection device also has a needle cover 15 with funnel-shaped, circumferentially extending, centering aid projecting radially outwards serving as a positioning element 15a, and a return mechanism 14 provided in the form of a helical spring which is biased by an axial pressure between the container holder 1 and the needle cover 15 in every axial position of the needle cover 15 in the assembled state. As with FIG. 1, but also visible in FIGS. 2 to 4, the needle cover 15 in the assembled state is accommodated in an annular gap between a distal (front or forward) portion of the container holder 1 and the casing structure 5 surrounding this distal portion, so that the needle cover 15 extends into the annular gap to a greater or lesser depth depending on its axial position in the proximal (rear or rearward) direction.

The exemplary container holder 1 is a simple cylindrical, e.g. circular cylindrical, sleeve, the hollow cross-section of which forms a housing chamber for a container containing a product or substance to be injected. The container may be provided in the form of a known medicament ampoule of the type used for accommodating insulin for a diabetic treatment or known from other treatments such as osteoporosis treatment. As may be seen from FIG. 2 for example, the container holder 1 forms a shoulder 1a projecting radially inward in the housing chamber at its distal or front end 3, which serves as a stop for a product container to be inserted. The housing chamber also serves as an axial slide guide for the container, which simultaneously also prevents any tilting movements of the inserted container. At its distal or front end 3, the container holder 1 has a slimmer cross-section than in the rest of its portion. It is provided with an external thread for a screw connection to a needle holder 10 at end 3. At the other, rear or proximal end, it is provided with another external thread, which provides the connection to a proximal housing part of the injection device. The proximal housing part or rear portion of the housing provides a mount for or carries a conveying mechanism for conveying the product and a dose metering unit which engages with the conveying mechanism for pre-setting a product dose to be dispensed by the conveying mechanism. Suitable conveying mechanisms and dose metering mechanisms have long been known and, therefore, require no further description. An outwardly projecting, peripheral shoulder 2 of the container holder 1 acts as a support for the spring 14 in the proximal direction.

The needle holder 10 which can be attached to the end 3 of the container holder 1 is pot-shaped with a sleeve part, which opens in the rearward direction and forms a base at its front end, through which an injection needle 11 axially extends. To obtain a connection of the needle holder 10 to the injection needle 11 across a larger axial length than that corresponding to the axial thickness of the base, a cylindrical projection extends out from the center of the base at its distal side, through which the injection needle 11 likewise extends. The injection needle 11 is firmly secured by the base of the needle holder 10 and the cylindrical projection. A distal needle portion projects freely from the distal end of the needle holder 10, i.e. from the cylindrical projection, forming a distal (forward or piercing) needle tip 3. A proximal needle portion projects out forming a proximal (rear piercing) needle tip in the hollow cross-section of the sleeve part of the needle holder 10, as may best be seen from FIGS. 2 to 4. The needle holder 10 is supplied by the manufacturer with an inner protective cap 12 and an outer protective cap 13. The inner protective cap 12 is fitted on the cylindrical projection of the needle holder 10. The outer protective cap 13 is fitted on the sleeve part of the needle holder 10. Once the outer protective cap 13 has been removed, the injection needle 11 is always completely surrounded by the inner protective cap 12 and is thus protected from damage while the user is protected from being pierced by the needle.

The needle cover is formed by a cylindrical, and in the embodiment illustrated as an example, a circular cylindrical, sleeve. It has a smooth external surface and an internal surface comprising two stages, as may be seen by comparing the drawings, for example FIG. 1 and FIG. 2. In its hollow cross-section, the needle cover 15 becomes wider from a proximal or rear portion in one stage to a distal or front portion. The distal and the proximal portions each have a cylindrical internal surface. Along the length of the proximal portion, a thickening is therefore obtained in which an annular gap 16 is formed, open rearwardly or in the proximal direction. In the assembled state, the annular gap 16 accommodates a distal portion of the spring 14 and serves as an axial guide for the spring 14. In the assembled state, the spring 14 is biased by a pressure between the annular shoulder 2 and the distal end of the annular gap 16 of the needle cover 15. Accordingly, the container holder 1 constitutes a proximal bearing for the spring 14, namely the annular shoulder 2, and the needle cover 15 serves as a distal thrust bearing, namely the distal end of the annular gap 16. The container holder 1 guides the axial sliding of the needle cover 15 in the assembled state. Its distal, or forward straight cylindrical portion surrounding the spring 14, illustrated in FIG. 1, serves as an axial slide guide for the container holder 1. The guide complementary surface is the internal surface of the proximal portion of the needle cover 15.

In the rear end or region of its proximal portion, the needle cover 15 is provided with a locking element. A resilient tongue 17 and an orifice 18 of the resilient tongue 17 co-operate to form the locking element. The resilient tongue 17 is integrally formed on the needle cover 15. The resilient tongue 17 is formed by two axial slits 19 in an external surface of the proximal portion of the needle cover 15, which extend as far as the proximal end of the needle cover 15. The resilient tongue 17 is elastically flexible in the radial direction and acts in the manner of a fixedly tensed flexing beam at one end, namely at its distal end.

The casing structure 5 forms a locking complementary element 8, as may be seen from FIG. 2, which extends through the orifice 18 when the needle cover 15 is in a proximal or rear end position and locks the needle cover 15 to prevent it from moving in the distal direction or forwardly by a form-fitting connection. The resilient tongue 17 with its orifice 18 and the locking complementary element 8 therefore establish a locking engagement based on a positive connection when the needle cover 15 is in the proximal end position. The positive connection exists between a stop surface of the locking complementary element 8 pointing in the proximal direction and a complementary stop surface facing this stop surface in the distal direction, formed by the resilient tongue 17 in the orifice 18.

To establish the locking engagement, the locking complementary element 8 is rigid, i.e. is not flexible. However, the locking engagement can be released due to the elastic flexibility of the locking element, because the locking element is bent elastically, radially inwardly, out of the locking engagement.

To release the locking engagement, i.e., bend the locking element, the injection device has an unlocking element 7. The unlocking element 7 is inserted in a recess 6 incorporated in the casing external surface of the needle cover 15 and extends through an orifice 9 formed in the region of the recess 6. The unlocking element 7 has an outwardly pointing pressure operating surface and a projecting extension, by which it extends radially inwards through the orifice 9 once inserted in the recess 6. The casing structure 5 forms the orifice 6 at such a point that when the injection device is in the assembled state, the orifice 6 is disposed radially exactly above the locking element. The orifice 9 is also designed so that it moves to a position lying above a distal end portion of the resilient tongue 17 in the assembled state. In the embodiment illustrated as an example, this distal end portion of the resilient tongue 17 is still disposed on the distal side of the orifice 18 so that the entire axial length of the resilient tongue 17 is able to bend elastically and the radial pressure force of the unlocking element 7 needed to produce the bending action is as low as possible.

To assemble the injection device, the container holder 1 and the casing structure 5 are joined to one another in the arrangement illustrated in FIG. 2, for example, so that the annular space for the needle cover 15 is left free between them. The annular space is closed by the annular shoulder 2 in the proximal or rear direction and is open in the distal or front direction. The needle cover 15 is then pushed into the annular space formed by the container holder 1 and the casing structure 5 with the spring 14 inserted in the annular gap 16 of the needle cover 15 as far as the proximal end position illustrated in FIG. 2. As it is pushed in, the resilient tongue 17 slides along the locking complementary element 8 and is thus bent elastically radially inwards until the orifice 18 radially overlaps or is aligned with the locking complementary element 8. As soon as this overlap or alignment is obtained, which is the case when the needle cover 15 is in the end position, the resilient tongue 17 snaps back elastically, radially outwardly, and the locking engagement illustrated in FIG. 2 is established.

Before pushing in the needle cover 15, the unlocking element 7 is inserted in the recess 6 of the casing structure 5 so that the extension of the unlocking element 7 extends through the orifice 9. The unlocking element 7 may be connected to the casing structure 5 by a frictional grip, but may be provided in the form of a clip-on connection at an axial end of the unlocking element 7. When connected to the casing structure 5, the unlocking element 7 is tensed at one end in the manner of an elastic flexing beam. The extension projecting radially inwardly through the orifice 9, which co-operates with the locking element of the needle cover 15 to release the locking engagement, is formed at the free end of this flexing beam or unlocking element 7. FIG. 2 illustrates how the unlocking element 7 is secured in the manner of a flexing beam. “D” denotes a compression force, by which the unlocking element 7 can be operated radially inwardly to release the locking engagement. The extension of the unlocking element 7 acting on the resilient tongue 17 to release the locking engagement is not in contact with the resilient tongue 17 when the unlocking element 7 is in the non-operating state, i.e. when the unlocking element 7 is not being operated, but is directed towards it at a slight radial distance from it.

Once the front part of the injection device and a rear part of the injection device incorporating the conveying mechanism and the dose metering mechanism have been joined to one another, the device is ready for sale. The user merely has to insert a container filled with a product to be injected in the container holder 1 and fit the needle holder 10. This is done the first time the device is used or whenever a used container has to be replaced. The needle holder 10 may also be replaced separately from when the container is changed.

In its proximal or rearwardly end position, the needle cover 15 lies with its distal end so far back behind the distal end 3 of the container holder 1 that the needle holder 10, which the user grips in the region of its sleeve part, can be comfortably connected to the container holder 1 by hand. Ideally, the needle cover 15 sits at least so far back behind the distal end 3 of the container holder 1 that at least a part, in some embodiments, the greater part, of the axial length of its sleeve part is exposed, permitting lateral access to the needle holder 10 when the latter is connected to the injection device. To release the needle holder 10, it may be gripped in the region where it is connected to the container holder 1, which is in the region of a screw thread providing a threaded engagement in the embodiment illustrated as an example. When the needle holder 10 is fitted, the injection needle 11 pierces a seal element closing a distal end of the product container accommodated in the container holder 1 by means of its proximal needle portion. A flow connection is therefore automatically established between the injection needle 11 and the interior of the product container when the needle holder 10 is fitted. Since the proximal end position of the needle cover 15 was selected so that the needle holder 10 can be fitted on the injection device and removed from the injection device when the needle cover 15 is fitted, the proximal or rearward end position will be referred to below as the fitting position of the needle cover 15. After fitting the needle holder 10, the inner protective cap 12 and the outer protective cap 13 are removed from the needle holder 10 with the needle cover 15 still in the fitting position, so that the injection needle 11 is exposed. Once the protective caps 12 and 13 have been removed, the locking engagement of the locking elements is released by depressing the unlocking element 7 and applying force in the direction indicated by D, so that the needle cover 15 moves in the forward direction under the effect of the elastic force of the tensed spring 14 until it reaches its protective position abutting against a stop. With the needle cover 15 in its protective position, the injection device is now ready for administering an injection.

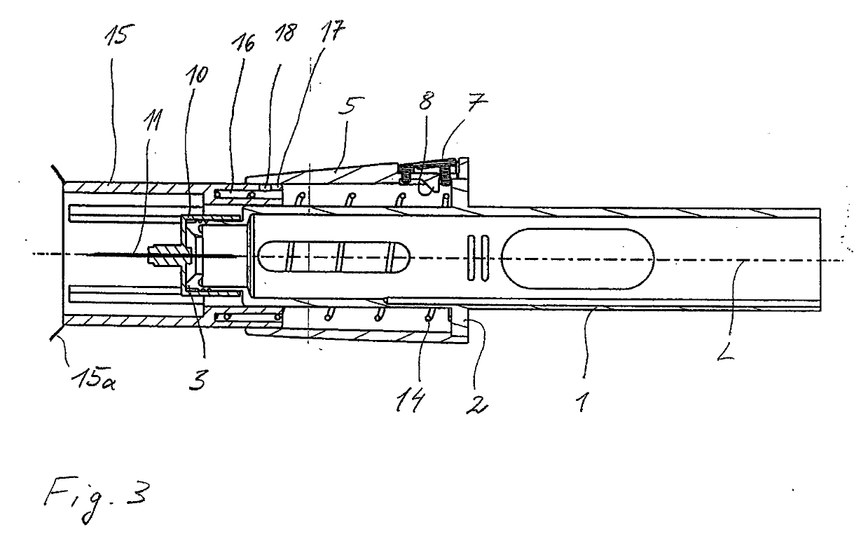

FIG. 3 illustrates the injection device with the needle cover 15 disposed in its protective position. In the protective position, the needle cover 15 extends beyond the tip of the injection needle 11. In the protective position, the needle cover 15 can be locked to prevent it from being pushed back in the proximal or rearward direction, preventing inadvertent access to the injection needle 11 from the side and thus reducing the risk of damage to the injection needle 11 and the risk of injury by the injection needle 11. Although the needle cover 15 is locked to prevent it from moving back in the protective position, this locking engagement can be released to administer an injection. If a locking engagement is not provided in the protective position, in which case the needle cover merely serves as a visual screen, the injection device with the needle cover 15 in such a protective position is immediately ready for the next injection. This is the case with the embodiment illustrated as an example.

In preparation for the injection, the user selects the dose of the product to be administered by means of the dose setting mechanism. The dose setting mechanism and the conveying mechanism are engaged in such a way that the product dose to be dispensed with the next injection is pre-defined by selecting the dose. Once the dose has been selected, the injection device is placed on the injection site by the distal or front end of the needle cover 15 being placed against the skin. By applying pressure in the distal direction, the injection device is moved in the distal direction relative to the needle cover 15. During the course of this movement, the needle cover 15 slides on the slide guide formed by the container holder 1 into the annular space between the container holder 1 and the casing structure 5 and, conversely, the injection needle 11 slides over the needle cover 15 in the distal direction into the body tissue. The piercing movement of the injection needle 11 ends when the distal end of the needle holder 10 arrives in contact with the skin surface and accordingly the distal or front end of the needle cover 15 and the distal or front end of the needle holder 10 are disposed axially adjacent. This state corresponds to the injection position of the needle cover 15.

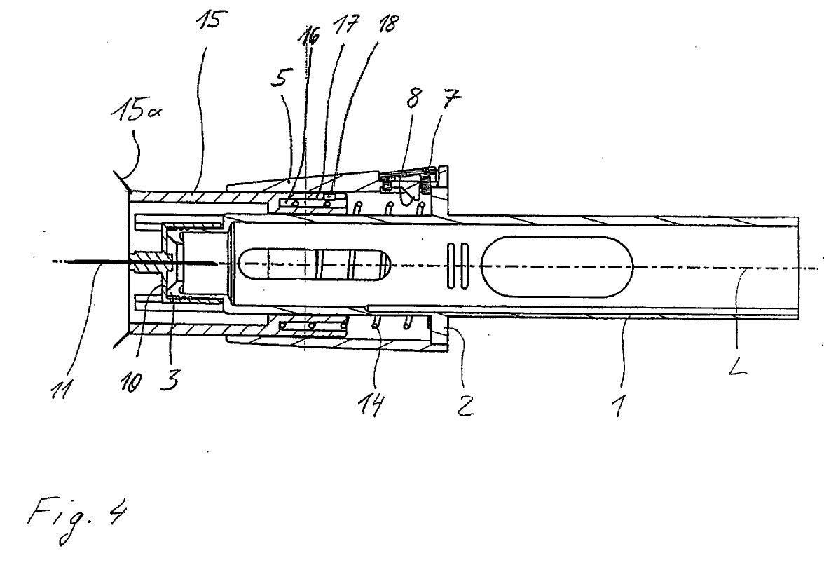

FIG. 4 illustrates the injection device with the needle cover 15 in its injection position. The injection needle 11 projects forwardly, with its entire free distal or forward needle portion extending beyond the needle cover 15. In the case of the described injection, it projects forward into the body tissue. In this state, the selected product dose is administered by the user, e.g. the patient, by operating the conveying mechanism. The conveying mechanism may comprise a plunger accommodated in the container, which can be moved axially in the distal direction, and a plunger rod acting on the plunger. Accordingly, to dispense and administer the product, the user pushes the plunger rod and hence the plunger in the distal or forward direction by a length which is pre-defined by the dose selection, as a result of which the selected product dose is dispensed from the container and administered through the injection needle 11. After administration, the injection needle 11 is pulled out of the body tissue. Since the needle cover 15 is held in the injection position solely by the opposing forces acting on the needle cover 15, namely the elastic force of the spring 14 and the pressure force opposing it, as the pressure force is released the needle cover 15 moves in the distal or forward direction back into the protective position under the action of the spring 14.

If the injection needle 11 has to be replaced, the needle cover 15 is pushed back beyond the injection position into a fitting position. In the last part of this movement, once the needle cover 15 has been moved in the rear or proximal direction beyond the injection position, the end of the resilient tongue 17 makes contact with the complementary locking element 8. The complementary locking element 8 tapers radially inwards at its side and, in the embodiment illustrated as an example, is directed with its end at an angle with respect to the axial direction. As a result of the tapered region of the locking element 8, the free end of the resilient tongue 17 is able to slide along the locking element 8 and is continuously bent radially inwardly as a result. As soon as the orifice 18 arrives at the same axial level as the locking element 8, the resilient tongue 17 snaps radially outwardly again due to it natural elastic force and establishes the locking engagement illustrated in FIG. 2. The needle holder 10 is freely accessible from the side again. Before removing the needle holder 10, at least one of the two protective caps 12 and 13 should be placed on the needle holder 10, to prevent injury by the injection needle 11. Only then should the needle holder 10 or the protective cap 13 placed on it, and the needle holder 10 be released from the container holder 1.

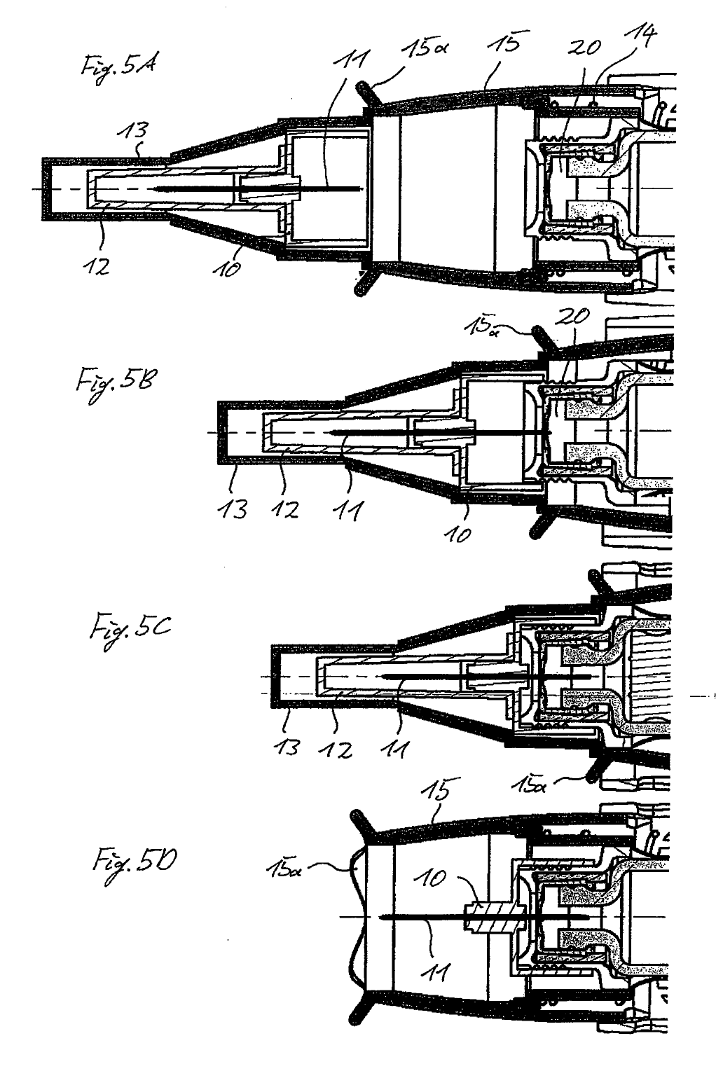

FIG. 5A is a cross-section of an embodiment of a positioning mechanism in accordance with the present invention comprising a needle cover 15 in the form of a sliding sleeve, which is connected to or mounted in an injection device in the manner described above. The sleeve has at its distal end a circumferentially extending edge projecting radially outwardly from the needle cover 15, which tapers, is angled, conically converges or is funnel-shaped, forming an extension of the distal opening of the needle cover 15. This serves as a positioning element 15a for positioning the needle holder 10 retained in the outer protective cap 13 on the needle cover 15. If the needle support or needle holder 10 retained in the outer needle cover 13 is fitted so that it is crooked relative to the needle cover 15, a force applied to the outer protective cap 13 more or less in the proximal direction of the injection device initially causes the circumferentially extending edge of the outer protective cap 13 to assume the position illustrated in FIG. 5A, being guided as it moves or slides along the edges 15a of the needle cover 15 standing out radially at an angle and serving as the positioning element. As a result, the needle holder 10 is oriented relative to the needle cover 15 in a specifically positioned or defined manner. When the user applies additional pressure to the retained outer protective cap 13 in the proximal direction of the injection device, the needle 11 oriented by the positioning elements 15a is moved towards the septum 20, guided in a centered position, and pierces it, as illustrated in FIG. 5B, so that the cannula 11 is not able to pierce the septum 20 at an angle. During the operation of pushing in the needle holder 10 and the subsequent full piercing of the septum 20 by the needle 11, illustrated in FIG. 5C, the needle cover 15 is pushed by the outer protective cap 13 in the proximal or rearwardly direction into the injection device against the force of the helical spring 14.

Once the needle holder 10 has been fitted and sits on the ampoule end, the outer protective cap 13 and hence also the inner protective cap 12 can be removed from the needle holder 10 and the needle 11. Since the outer protective cap 13 has been removed, the needle cover 15 is moved forwardly in the distal direction beyond the needle 11 due to the distally or forwardly directed pre-tensioning, so that exposed needle 11 is protected when the inner protective cap 12 is removed.

As may be seen from FIG. 5D, the positioning element 15a may comprise several positioning elements 15a extending around the distal opening of the needle cover 15, which may be in the shape of flower petals when seen in a plan view onto the injection device from the distal direction, for example.

Starting from the state illustrated in FIG. 5D, an injection can be administered, in which case the needle cover 15 extracted beyond the needle 11 is placed with the distal, forward or free end on an injection site. The injection device with the injection needle 11 connected to it is pushed against the injection site, as a result of which the needle cover 15 is pushed back proximally or rearwardly into the injection device to expose the needle 11. Once the injection has been administered, the injection device can be removed form the injection site again, as a result of which the needle cover 15 automatically slides back over the needle 11 to protect it.

To remove the needle 11 retained in the needle holder 10 from the injection device, the outer protective cap 13 may be placed back on the needle cover 15, in which case it will be centered by the positioning elements 15a and positioned by pushing the needle cover 15 in, so that it is guided, towards the needle 11 and the needle holder 10 and latching or screwing it to the needle holder 10. Thus, when removing the needle holder 10 from the injection device, the needle 11 is covered by the outer protective cap 13 during the removal operation, thereby eliminating any risk of injury.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A positioning mechanism for placing a needle in a piercing position on an injection device, the positioning mechanism comprising:
one of a needle guide, a needle cover, a needle holder or a protective cap for the needle which is or can be detachably mounted on the injection device, and
at least one positioning element associated with the one of a needle guide, needle cover, needle holder or protective cap adjacent to an end thereof, whereby the needle is placed in the piercing position by the positioning element.

2. The positioning mechanism according to claim 1, comprising at least one surface element such that an intersection curve between the surface element and a plane extending through a positioning axis of the needle extends towards the positioning axis of the needle.

3. The positioning mechanism according to claim 1, wherein the at least one positioning element extends at least partially around the distal end of the one of a needle guide, needle cover, needle holder or protective cap.

4. The positioning mechanism according to claim 1, wherein the at least one positioning element comprises a conically tapering portion.

5. The positioning mechanism according to claim 1, wherein the at least one positioning element comprises a portion extending radially with respect to the needle cover.

6. The positioning mechanism according to claim 2, wherein a portion of the surface element extends an angle of 30° to 60° or 40° to 50° with respect to the positioning axis.

7. The positioning mechanism according to claim 1, wherein the one of a needle guide, needle cover, needle holder or protective cap is moveable in an axial direction relative to the injection device.

8. An injection device comprising a needle cover, the injection device comprising:
a) a housing for receiving a container containing an injectable product or the container itself,
b) a needle holder comprising an injection needle connectable to the injection device or container by the needle holder, and
c) a needle cover carried by the housing such that it can be moved against a restoring force from a protective position, beyond an injection position into a fitting position, wherein
d) the needle cover extends beyond a needle tip of the injection needle in the protective position and is short of the needle tip in the injection position, wherein the needle cover affords free access to the needle holder in the fitting position, such that the needle holder can be held by a protective sleeve and can be connected, to or released from the housing, and wherein the needle cover comprises a centering aid at the one end, onto which an outer protective cap surrounding the injection needle can be fitted and orientated relative to the injection device or container.

9. A method for fitting a needle on an injection device or on an ampoule in a positioned manner, the method comprising the steps of;
providing a needle holder carrying a needle or a needle;
providing a positioning element associated with a needle cover connectable to the injection device or to the ampoule;
positioning the needle relative to the needle cover and, thus, relative to the injection device or the ampoule.

10. The method according to claim 9, further comprising moving the needle cover toward the injection device or the ampoule, the needle holder or the needle thus guided onto the injection device or the ampoule in a positioned manner.
